(19) United States
(12) Patent Application Publication
(10) Pub. No.: US 2008/0181975 A1
(43) Pub. Date: Jul. 31, 2008

(54) Vasoprotective agent

(75) Inventors: Naonobu Yoshizuka, Tochigi (JP); Yuko Fukuda, Tochigi (JP); Yasuko Amano, Tochigi (JP); Minoru Takizawa, Tochigi (JP); Hajime Soga, Tokyo (JP); Shinobu Mori, Tokyo (JP); Akira Hachiya, Cincinnati, OH (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Kao Corporation, Tokyo (JP)

(21) Appl. No.: 11/700,200

(22) Filed: Jan. 31, 2007

(51) Int. Cl. A61K 36/752; A61K 36/00; A61K 36/16; A61K 36/81; A61P 7/00; A61K 36/42; A61K 36/537

(52) U.S. Cl. 424/736; 424/746; 424/752; 424/758; 424/760; 424/773

(57) ABSTRACT

This invention provides a vasoprotective agent containing a plant or its extract as an effective ingredient. The plant is selected from the group consisting of rosemary, sage, geranium herb, adlai, field horsetail, bitter orange peel, fucus, burdock, dokudami, Japan pepper, Ophiopogon tuber, ginkgo, natsume and dishcloth gourd.

### TECHNICAL FIELD OF THE INVENTION

This invention relates to a vasoprotective agent, which inhibits weakening of the blood vessel and strengthens the blood vessel.

### BACKGROUND OF THE INVETION

The onset of a peripheral vascular disease, such as spidervein, varix, rosacea, telangiectasia, purpura, epistaxis, fundal hemorrhage or chronic venous insufficiency, is known to stem from weakening of the blood vessel such as a reduction in the mechanical strength of vascular tissue and/or an increase in capillary permeability. It is also known that, if a healthy person exhibits a reduction in the mechanical strength of vascular tissue or increases capillary permeability because of standing or sitting for long time, such conditions could lead to the swelling of the lower half of the body, especially the swelling of the legs, a feeling of fatigue and swelling in the legs alike. Therefore, a vasoprotective agent that improves the mechanical strength of vascular tissue and the resistance of capillaries is thought to be effective for the prevention, alleviation or treatment of such diseases.

Bioflavonoids such as hesperidin and rutin have already been reported to be useful for venous insufficiency in the lower limb, pile and the like (C. Allegra, et al., Lymphology, 31(suppl), 12-16 (1998)) and also to be useful for swelling (JP-A-10-182468, JP-A-10-218777) . Because bioflavonoids are traditionally known to have a safety problem in the case of transdermal application, there are an outstanding desire for the provision and use of safer and more effective medicines.

### SUMMARY OF THE INVENTION

Described specifically, the present invention relates to the following aspects 1) to 6):
1) A vasoprotective agent containing, as an effective ingredient, a plant, which is selected from the group consisting of rosemary, sage, geranium herb, adlai, field horsetail, bitter orange peel, fucus, burdock, dokudami, Japan pepper, ophiopogon tuber, ginkgo, natsume and dishcloth gourd, or an extract thereof.
2) A vascular endothelial cell contraction inhibitor containing, as an effective ingredient, a plant, which is selected from the group consisting of rosemary, sage, geranium herb, adlai, field horsetail, bitter orange peel, fucus, burdock, dokudami, Japan pepper, Ophiopogon tuber, ginkgo, natsume and dishcloth gourd, or an extract thereof.
3) A method for the prevention, alleviation or treatment of a disease caused by weakening of the blood vessel, which comprises administering an effective dose of a plant, which is selected from the group consisting of rosemary, sage, geranium herb, adlai, field horsetail, bitter orange peel, fucus, burdock, dokudami, Japan pepper, ophiopogon tuber, ginkgo, natsume and dishcloth gourd, or an extract thereof.
4) A method for the prevention or alleviation of swelling, which comprises administering an effective dose of a plant, which is selected from the group consisting of rosemary, sage, geranium herb, adlai, field horsetail, bitter orange peel, fucus, burdock, Dokudami, Japan pepper, Ophiopogon tuber, ginkgo, Natsume and dishcloth gourd, or an extract thereof.
5) A method for the prevention or alleviation of spider vein, which comprises administering an effective dose of a plant, which is selected from the group consisting of rosemary, sage, geranium herb, adlai, field horsetail, bitter orange peel, fucus, burdock, dokudami, Japan pepper, Ophiopogon tuber, ginkgo, natsume and dishcloth gourd, or an extract thereof.
6) A method for the prevention or alleviation of tired leg, which comprises administering an effective dose of a plant, which is selected from the group consisting of rosemary, sage, geranium herb, adlai, field horsetail, bitter orange peel, fucus, burdock, dokudami, Japan pepper, Ophiopogon tuber, ginkgo, natsume and dishcloth gourd, or an extract thereof.

### DETAILED DESCRIPTION OF THE INVENTION

The present invention relates to a vasoprotective agent, which exhibits excellent vascular protection effect and is useful as medicines and/or cosmetics.

The present inventors conducted an investigation into an assessment system for vasoprotective agents. As a result, it was found that, when vascular endothelial cells were seeded on collagen gel, the collagen gel was contracted by the force of the cells. It has also been found that the assessment of a contraction inhibiting substance for vascular endothelial cells or a capillary-wall strengthening substance is feasible by using as an index the phenomenon observed that the area of the gel shrinks. Further, it has also been found that the vascular protection effect or contraction inhibiting effect for vascular endothelial cells is found in particular plants and extracts thereof, and such particular plants and extracts thereof are effective for the prevention, alleviation or treatment of diseases caused by the fragility of blood vessels and also for the alleviation of the symptoms of swollen or tired leg.

The vasoprotective agent and the vascular endothelial cell contraction inhibitor according to the present invention exhibit excellent vascular protection effect, and are effective as medicines and/or cosmetic preparations for the prevention, alleviation or treatment of peripheral vascular diseases caused by the weakening of the blood vessels. They are also useful as medicines and/or cosmetic preparations for the alleviation of the symptoms of swelling, tired leg (a feeling of fatigue, swelling or the like in the legs) or the like, which is caused by a reduction in vascular resistance and an increase in vascular permeability as a result of temporary weakening of blood vessels.

In the present invention, the term “rosemary” means Rosmarinus officinalis of the family Labiatae, the term “sage” means Salvia officinalis or Salvia splendens and the like of the family Labiatae, the term “geranium herb” means Geranium thunbergii Siebold et Zuccarini of the family Geraniaceae, the term “adlai” means Coix lachryma-jobi Linne var. ma-yuen Stapf of the family Gramineae, the term “field horsetail” means Equisetum arvense Linne of the family Equisetaceae, the term “bitter orange peel” means Citrus aurantium Linne or Citrus aurantium Linne var. daidai Makino of the family Pinaceae, the term “fucus” means Fucus Vesiculosis of the family Fucaceae, the term “burdock” means Arctium lappa Linne of the family Compositae, the term “dokudami” means Houttuynia cordata Thunbergof the family Saururaceae, the term “Japan pepper” means Zanthoxylum piperi tum De Candolla or another plant of the same genus of the family Rutaceae, the term “Ophiopogon tuber” means Ophiopogon japonica or another plant of the same genus of the family Liliaceae, the term “ginkgo” means Ginkgo biloba Linne of the family Ginkgoaceae, the term “natsume” means Zizyphus jujuba MILL. var. inermis (BUNGE) REHD. of the family Rhamnaceae, and the term “dishcloth gourd” means Luffa cylindrica M. Reomen or Luffa cylindrica of the family Cucurbitaceae.

As each plant described above, its whole part, leaves, flowers, barks, branches, fruits, roots or the like can be used as are or after grinding them. However, in a case of rosemary, it is preferred to use its leaves or flowers; in a case of sage, its leaves or flowers; in a case of geranium herb, its aerial parts; in a case of adlai, its seeds; in a case of field horsetail, the whole plant; in a case of bitter orange peel, its pericarp; in a case of fucus, the whole alga; in a case of burdock, its roots; in a case of dokudami, its aerial parts; in a case of Japan pepper, its pericarp; in a case of ophiopogon tuber, its roots; in a case of ginkgo, its leaves; in a case of natsume, its fruits; and in a case of dishcloth gourd, its aerial parts or the whole plant.

The term “extract” as used herein means an extract obtained by extracting the above-described plant at room temperature or under heating or in an extraction apparatus such as Soxhlet extract or with any of various solvents; or its dilution, concentrate or dried powder.

As an extraction solvent usable to obtain the plant extract of the present invention, either a polar or nonpolar solvent can be used. Illustrative are water; alcohols such as methanol, ethanol, propanol and butanol; polyhydric alcohols such as propylene glycol and butylene glycol; ketones such as acetone and methyl ethyl ketone; esters such as methyl acetate and ethyl acetate; linear or cyclic ethers such as tetrahydrofuran and diethyl ether; polyethers such as polyethylene glycol; hydrocarbons such as squalane, hexane, cyclohexane and petroleum ether; aromatic hydrocarbons such as toluene; halogenated hydrocarbons such as dichloromethane, chloroform and dichloroethane; and carbon dioxides. They can be used either singly or in combination.

The plant extract may be used as is. As an alternative, the plant extract may also be used by preparing it into a powder or paste form subsequent to diluting or concentrating it or subjecting it to freeze-drying.

Further, the plant extract may also be used after removing inactive impurities from the same by a separation technology such as liquid-liquid partition chromatography. It is to be noted that two or more plants or plant extracts may be used together.

The present inventors found that, when vascular endothelial cells are seeded on collagen gel, the collagen gel is contracted by the force of the cells. This is believed to mean that functions, fragility and strength of the blood vessels, which contain vascular endothelial cells as one of their constituents, can be measured by relying upon the contraction phenomenon of collagen as an index. Described specifically, vascular endothelial cells are considered to be producing tension at their surfaces by controlling the tension of cytoskeleton-forming α-actin such that the cells retain their own shapes. Also known is the phenomenon that, when thrombin is caused to act, α-actin coagulates to produce still greater tension and as a result, the cells themselves contract. It is also known that vascular endothelial cells strongly adhere to extra cellular matrix molecules such as collagen and fibronectin via adhesion molecules or crosslinking molecules such as integrin. Induction of cell contraction by the production of tension by skeletal molecules in vascular endothelial cells results in the application of an action in which the adhered extracellular matrix substance is pulled together. Seeding of vascular endothelial cells on a material body with collagen gel solidified to such a large size as permitting a visual observation, therefore, makes it possible to observe a contraction phenomenon of the collagen gel caused by the tension of the vascular endothelial cells, and further, the addition of a stimulus with thrombin or the like causes cell contraction, thereby making it possible to promotively induce the contraction of the collagen gel under still greater tension.

In a blood vessel in the living body, specifically in a tissue such as the skin or blood-brain barrier, on the other hand, it has been anatomically indicated that vascular endothelial cells are close to each other and are in the form of a semipermeable membrane with the cells blocked together in a single layer. Between blood in the blood vessel and water in the extravascular tissue, control is effected such that by using as a primary route the spaces between the vascular endothelial cells, water and low-molecular nutrients permeate but high-molecular substances as blood components hardly permeate. It is considered that, once the controlling action on the spaces between the vascular endothelial cells fails and the spaces become wider, the permeation of substances dramatically increases to result in the permeation of even blood components. Contraction of the individual cells is believed to take large part in such failure of the controlling action on the intercellular spaces and space widening. A substance that inhibits the contraction phenomenon of cells is, therefore, considered to show an effect to enhance the ability to retain the structure of blood vessels, that is, vascular protection effect in the living body. It is, hence, possible to assess a contraction inhibiting substance for vascular endothelial cells or a vasoprotective agent by using, as an index, the phenomenon observed that an area of gel shrinks.

The plants and their extracts, which are useful in the present invention, inhibit the above-described contraction of collagen gel as will be described subsequently in Examples. They are, thus, believed to be equipped with vascular protection effect and also with contraction inhibiting effect for vascular endothelial cells.

Accordingly, these plants and extracts can be formulated, as intercellular protectants or vascular endothelial contraction inhibitors, into medicines or cosmetic preparations effective for the prevention, alleviation or treatment of diseases caused by weakening of blood vessels, for example, peripheral vascular diseases such as spider vein, varix, rosacea, telangiectasia, purpura, epistaxis, fundal hemorrhage and chronic venous insufficiency; or into medicines or cosmetics for the alleviation of symptoms of swelling of the lower half of the body, swollen leg, tired leg (a feeling of fatigue or swelling in the legs) and the like as caused by the occurrence of an imbalance between the drainage of body fluid from capillaries and the collection of body fluid by lymphatic system as a result of temporary weakening of blood vessels, a reduction in vascular resistance and an increase in vascular permeability.

When the plant or extract useful in the present invention is employed as a medicine, examples of its preparation form include internal preparations such as tablets, capsules, granules and syrups; injections; suppositories; inhalants; transdermal absorption preparations; and external preparations such as ointments, solutions, extracts, lotions and emulsions, with the use in the form of external preparations being preferred. In these pharmaceutical preparations, pharmaceutically acceptable additives such as preparation aids, stabilizers, humectants, emulsifiers, absorbefacients and surfactants may be added in a desired combination in addition to the plant or its extract useful in the present invention.

When the plant or extract useful in the present invention is employed as a cosmetic preparation, it can be formulated into various forms, for example, water-in-oil or oil-in-water type emulsified cosmetic preparations, creams, lotions, gels, foams, essences, foundations, packs, sticks, powders, granules, ointments, milky lotions and soon. In these cosmetic preparations, cosmetic oils, surfactants, ultraviolet light absorbers, alcohols, chelating agents, pH adjustors, preservatives, viscosity increasing agents, colorants, fragrance ingredients, various skin nutrients and the like, which are commonly used as cosmetic ingredients, may be added in a desired combination in addition to the plant or its extract useful in the present invention.

The content of the above-described plant or its extract in the medicine or cosmetic preparation according to the present invention may be set preferably at from 0.002 to 20 wt %, more preferably from 0.01 to 10 wt % in terms of dry weight when the plant is employed. In the case of the extract, on the hand, its content may be set at preferably from 0.0002 to 2 wt %, more preferably from 0.001 to 1 wt % in terms of solid content.

When used as a medicine, its dose is generally from 1 to 10,000 mg, preferably from 10 to 5,000 mg per adult in terms of the plant or extract useful in the present invention (on a dry weight basis) , although it varies depending on the body weight, age, sex and conditions of each patient, its administration route and frequency, etc.

### EXAMPLES

### Example 1

### Assessment Method of Collagen Gel Contraction Inhibiting Effect

A collagen gel solution for a three-dimensional culture kit for vascular endothelial cells (Iwaki Scitech) was prepared by conducting mixing in accordance with the formulation. After the collagen gel solution was poured at 500 μL/well into a 24-well microplate (189 mm²/well), incubation was conducted at 37° C. for 30 minutes or longer to solidify the collagen gel solution. On the resultant gel, human umbilical vein endothelial cells (HUWEC) were seeded at a population of 0.7×10⁵ cells/well to immobilize the cells. At the time of the seeding, a culture medium with bovine serum (Kanto Chemical) added at 10% in a vascular endothelial cell basal medium, EBM-2 (Clonetics), was used. Twenty-four hours after the seeding, the medium in each culture well was replaced with a serum-free medium (EBM-2) in which the corresponding one of the plant extracts shown in Table 1 had been added, and further, the gel was separated from the wall of the well and was subjected to suspension culture. As a control, EBM-2 was used without addition of any plant extract. Subsequent to culture for 17 hours, the medium in each well was replaced with EBM-2 which contained 0.5 U/mL of thrombin (Sigma). Thirty minutes later, the upper diameter of each contracted gel was measured to calculate the area of the gel. For the assessment of effectiveness, the contraction inhibition rate (%) of each plant extract was determined. Specifically, a difference in gel area between each plant-extract-added sample and the control was expressed in terms of a percentage based on a gel-area contraction in the control (the following formula 1).

$$Contraction inhibition rate ( \% ) = \frac{\begin{matrix}( Area of plant \text{-} extract \text{-} added sample after contraction ) - \\ ( Area of control after contraction )\end{matrix}}{\begin{matrix}( Area of plant \text{-} extract \text{-} added sample before contraction ) - \\ ( Area of control after contraction )\end{matrix}} \times 100 \qquad ( Formula 1 )$$

TABLE 1
Examples

| Names of extracts | Contraction inhibition rate (%) |
|---|---|
| Rosemary | 68 |
| Sage | 66 |
| Geranium herb | 23 |
| Adlai | 11 |
| Field horsetail | 11 |
| Bitter orange peel | 9 |
| Fucus | 7 |
| Burdock | 7 |
| Dokudami | 5 |
| Japan pepper | 5 |
| Ophiopogon tuber | 3 |
| Ginkgo | 3 |
| Natsume | 2 |
| Dishcloth gourd | 2 |

Details of the plant extracts employed in Example 1 are as shown in Table 2. They were each prepared in a manner known per se in the art.

TABLE 2

| | | | Solid |
|---|---|---|---|
| | | Extraction | contents |
| Names of extracts | Used parts | solvent | (w/v %) |
| Rosemary | Leaves | 50% Ethanol | 2.2 |
| Sage | Leaves | 50% Ethanol | 2.6 |
| Geranium herb | Aerial parts | 50% Ethanol | 1.8 |
| Adlai | Seeds | 50% Ethanol | 0.2 |
| Field horsetail | Whole plant | 50% Ethanol | 1.6 |
| Bitter orange peel | Pericarp | 50% Ethanol | 3.7 |
| Fucus | Whole alga | 50% Ethanol | 0.6 |
| Burdock | Roots | 50% Ethanol | 1.7 |
| Dokudami | Aerial parts | 50% Ethanol | 0.8 |
| Japan pepper | Pericarp | 50% Ethanol | 0.8 |
| Ophiopogon tuber | Roots | 50% Ethanol | 9.2 |
| Ginkgo | Leaves | 50% Ethanol | 2.7 |
| Natsume | Fruits | 50% Ethanol | 5.6 |
| Dishcloth gourd | Aerial parts | 50% Ethanol | 0.6 |

### Example 2

### Alleviation of Swollen Leg, Alleviation of Spider Vein, Alleviation of Tired Leg, and Assessment Methods

Using 26 females aged from 25 to 45 as subjects in a rosemary group and those in a control group, respectively, a text was conducted. The 13 subjects in the rosemary group applied the lotion shown as an example product in Table 3 to the legs in the morning and evening for a predetermined period, while the 13 subjects in the control group likewise applied a control lotion of a similar formulation as the example product except for the substitution of purified water for the rosemary extract. The severities of swollen leg, leg spider vein and tired leg in each group were assessed.

In the morning and afternoon before the initiation of the application and also in the morning and afternoon after the repeated use for 8 weeks, the leg (calf) volume of each subject was measured by a three-dimensional digitizer (Vivid 900, manufactured by Konica Minolta). Swollen leg was assessed based on the percentage of the leg volume in the afternoon to the leg volume in the morning. The results are shown in Table 4.

Concerning leg spider vein, spider veins in the legs of each subject were photographed. The degree of alleviation upon elapsed time of 4 weeks after the initiation of the application was assessed in accordance with the assessment standards in Table 5, and the results are shown in Table 6.

The degree of alleviation of tired leg in each subject was assessed 4 weeks after the initiation of the application in accordance with the assessment standards in Table 7 based on a questionnaire to her, and the results are shown in Table 8.

TABLE 3
Example product

| Composition | Contents (wt %) |
|---|---|
| Rosemary extract | 5.00 |
| Glycerin | 6.00 |
| Petrolatum vaseline | 2.00 |
| Stearyl alcohol | 2.31 |
| Cetyl alcohol | 0.99 |
| Mineral oil | 1.50 |
| Stearic acid | 0.50 |
| Acrylic acid polymer | 0.13 |
| Polyoxyethylene ether | 1.35 |
| Glyceryl dilaurate | 0.50 |
| Modified starch | 1.00 |
| Silicone oil | 1.50 |
| Preservative | 0.70 |
| Sodium hydroxide | 0.10 |
| Purified water | Balance |

TABLE 4
Swollen Leg Alleviation Effect

| | Before* | After** |
|---|---|---|
| | repeated use | repeated use |
| Rosemary group | 101.3 ± 1.2% | 100.8 ± 0.8% |
| Control group | 101.2 ± 1.4% | 102.0 ± 1.5% |

*Swelling before repeated use = Leg volume in the afternoon at the time of initiation of application/Leg volume in the morning at the time of initiation of application
**Swelling after repeated use = Leg volume in the afternoon after 8 weeks/Leg volume in the morning after 8 weeks

As evident from the above-described results, the rosemary group was more pronounced in swollen-leg alleviation effect than the control group.

TABLE 5
Degree of Spider Vein Alleviation

| | Degree of spider |
|---|---|
| Score | vein alleviation |
| 7 | Pronoucedly alleviated |
| 6 | Alleviated |
| 5 | Slightly alleviated |
| 4 | No change |
| 3 | Slightly deteriorated |
| 2 | Deteriorated |
| 1 | Pronoucedly deteriorated |

TABLE 6
Spider Vein Alleviation Effect

| | Alleviation score |
|---|---|
| Rosemary group | 4.2 ± 1.1 |
| Control group | 3.4 ± 1.2 |

As evident from Table 6, the rosemary group was higher in spider vein alleviation score than the control group.

TABLE 7
Assessment Standards for Tired Leg Alleviation

| Score | Assessment standards |
|---|---|
| 5 | Very satisfied |
| 4 | Satisfied |
| 3 | Neither satisfied nor dissatisfied |
| 2 | Dissatisfied |
| 1 | Very dissatisfied |

TABLE 8
Tired Leg Alleviation Effect

| | Alleviation score |
|---|---|
| Rosemary group | 3.4 ± 1.1 |
| Control group | 2.9 ± 0.7 |

As evident from Table 8, the example product has been found to have higher tired leg alleviation effect than the control.

1. A vasoprotective agent comprising as an effective ingredient a plant, which is selected from the group consisting of rosemary, sage, geranium herb, adlai, field horsetail, bitter orange peel, fucus, burdock, dokudami, Japan pepper, Ophiopogon tuber, ginkgo, natsume and dishcloth gourd, or an extract thereof.

2. A vascular endothelial cell contraction inhibitor comprising as an effective ingredient a plant, which is selected from the group consisting of rosemary, sage, geranium herb, adlai, field horsetail, bitter orange peel, fucus, burdock, dokudami, Japan pepper, ophiopogon tuber, ginkgo, natsume and dishcloth gourd, or an extract thereof.

3. A method for the prevention, alleviation or treatment of a disease caused by weakening of the blood vessel, which comprises administering an effective dose of a plant, which is selected from the group consisting of rosemary, sage, geranium herb, adlai, field horsetail, bitter orange peel, fucus, burdock, dokudami, Japan pepper, ophiopogon tuber, ginkgo, natsume and dishcloth gourd, or an extract thereof.

4. The method according to claim 3, wherein said disease caused by said weakening is a peripheral vascular disease selected from the group consisting of spidervein, varix, rosacea, telangiectasia, purpura, epistaxis, fundal hemorrhage and chronic venous insufficiency.

5. A method for the prevention or alleviation of swelling, which comprises administering an effective dose of a plant, which is selected from the group consisting of rosemary, sage, geranium herb, adlai, field horsetail, bitter orange peel, fucus, burdock, dokudami, Japan pepper, Ophiopogon tuber, ginkgo, natsume and dishcloth gourd, or an extract thereof.

6. A method for the prevention or alleviation of spider vein, which comprises administering an effective dose of a plant, which is selected from the group consisting of rosemary, sage, geranium herb, adlai, field horsetail, bitter orange peel, fucus, burdock, dokudami, Japan pepper, Ophiopogon tuber, ginkgo, natsume and dishcloth gourd, or an extract thereof.

7. A method for the prevention or alleviation of tired leg, which comprises administering an effective dose of a plant, which is selected from the group consisting of rosemary, sage, geranium herb, adlai, field horsetail, bitter orange peel, fucus, burdock, dokudami, Japan pepper, ophiopogon tuber, ginkgo, natsume and dishcloth gourd, or an extract thereof.
